(19) United States
(12) Patent Application Publication
(10) Pub. No.: US 2008/0178873 A1
(43) Pub. Date: Jul. 31, 2008

(54) DEVICE FOR PREVENTION OF SNORING

(76) Inventor: Adam L. Alpers, Ocala, FL (US)

Correspondence Address: NYDEGGER & ASSOCIATES, 348 OLIVE STREET, SAN DIEGO, CA, 92103, US

(21) Appl. No.: 11/627,266

(22) Filed: Jan. 25, 2007

(51) Int. Cl. A61M 15/00

(52) U.S. Cl. 128/200.24

(57) ABSTRACT

A nasal dilator is inserted into a user's nostrils to facilitate breathing and eliminate snoring. In overview, the nasal dilator includes a bridge and two dilation members. Structurally, the bridge is U-shaped and terminates at a first end and a second end. For purposes of the invention, a dilation member is attached to each end of the bridge. Further, each dilation member is tubular and is flexibly biased toward a substantially cylindrical configuration. During use, each dilation member engages a nasal wall and urges the nasal wall radially outward to facilitate breathing.

### FIELD OF THE INVENTION

The present invention pertains generally to systems and methods for facilitating nasal breathing. More particularly, the present invention pertains to systems and methods for enlarging the cross-sectional flow area of the nostrils. The present invention is particularly, but not exclusively, useful as a system for facilitating nasal breathing through positioning a device in the nostrils to enlarge the breathing passageways to eliminate snoring.

### BACKGROUND OF THE INVENTION

Many people suffer from some malformation of the nasal passages which makes nasal breathing difficult or which leads to snoring. For instance, such a malformation may include a deviated septum, injured bone structure, or swelling due to allergies or illness. Medically, the lower portion of the nostril immediately above the entrance to the nostril is known as a nasal vestibule. In properly formed noses, the vestibule tapers inwardly to a narrowed neck-like area called the ostium internum. Above the ostium internum the nasal passages widen. Nasal obstructions commonly occur at the ostium in individuals who have swelling due to allergies, a deviated septum or similar condition. Commonly, the lateral wall at the ostium is loose with the result that the outer wall tissue draws in during the process of inhalation to substantially block the passage of air through the nasal passage. Also, a loose lateral wall at the ostium can increase the likelihood and severity of snoring.

Often, blockages of the nasal passages are frustrating. In particular, when the nasal passages are blocked, sustained mouth breathing over a long period of time may cause lung irritation due to the inhalation of foreign particles that would otherwise be filtered if the breath had been passed through the nose. Blockage of the nasal passages is particularly uncomfortable at night, since it is uncomfortable for many people who have such a problem to breathe through the mouth while asleep. Nasal blockage can lead to sleep disturbances and irregularities because those with such a condition may often wake during the night because of oxygen depletion. Further, snoring may wake the sufferer or others.

The most common approach to a serious and chronic nasal blockage problem as described above is a surgical attempt to correct the malformation of the nasal passages. However, surgery is expensive and may not ultimately correct the problem.

In light of the above, it is an object of the present invention to provide a system and method for facilitating nasal breathing and eliminating snoring. It is another object of the present invention to provide a system for enlarging the nasal passageways. It is another object of the present invention to provide a system in which nasal walls are urged radially outward from the nasal passageways by system elements inserted into the nostrils. It is yet another object of the present invention to provide a system and method for facilitating nasal breathing which ensures that system elements inserted into the nostrils do not fall out. Yet another object of the present invention is to provide a system for facilitating nasal breathing which is easy to use, relatively simple to implement, and comparatively cost effective.

### SUMMARY OF THE INVENTION

The present invention is directed to a nasal dilator to facilitate breathing and eliminate snoring. More specifically, the dilator is designed for insertion into a user's nostrils. Once inserted, the dilator urges the nasal walls radially outward to enlarge the nasal passageways and increase air flow. Structurally, the nasal dilator includes a substantially U-shaped bridge connected to two dilation members which provide the radially outward force on the nasal walls.

In greater structural detail, the bridge includes a first leg and a second leg that are interconnected by an integral handle member. Each leg terminates at an end that is connected to a respective dilation member. Further, each leg defines a respective leg axis, with the axes defining a plane. In order to ease insertion of the dilation members into the nostrils, the handle member includes a flex axis that is perpendicular to the plane defined by the leg axes.

For purposes of the present invention, each dilation member is tubular and forms an aperture. Further, each aperture defines an aperture axis that is substantially parallel to the adjacent leg's axis. Also, each dilation member has a length extending in a direction parallel to the respective aperture axis from a substantially planar proximal edge to a distal edge. Each tubular dilation member has a peri-septum side and an apo-septum side. For attachment between each dilation member and the bridge, each end of the bridge is attached to the peri-septum side of each dilation member. Importantly, each dilation member is flexibly biased toward a substantially cylindrical configuration to urge the nasal walls radially outward.

In order to facilitate breathing, a user grips the handle and inserts each dilation member into a respective nostril. Upon insertion into the respective nostril, each dilation member engages a respective nasal wall to urge the respective nasal wall radially away from the respective aperture axis to facilitate breathing. Further adjustment of the position of each dilation member for the user's comfort is accomplished by manipulating the handle. When the nasal dilator is comfortably positioned, the bridge prevents non-axial movement of the dilation members within the nostrils. Further, the engagement between the dilation members and the nostrils prevents axial movement of the nasal dilator, i.e., the nasal dilator does not fall out of the nose.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a nasal dilator positioned in a user's nostrils to facilitate breathing in accordance with the present invention;

FIG. 2 is an enlarged perspective view of the nasal dilator of FIG. 1;

FIG. 3 is a front view of the nasal dilator of FIG. 2;

FIG. 4 is a side view of the nasal dilator of FIG. 2; and

FIG. 5 is an end view of a dilation member of the nasal dilator of FIG. 2.

### DESCRIPTION OF THE PREFERRED EMBODIMENTS

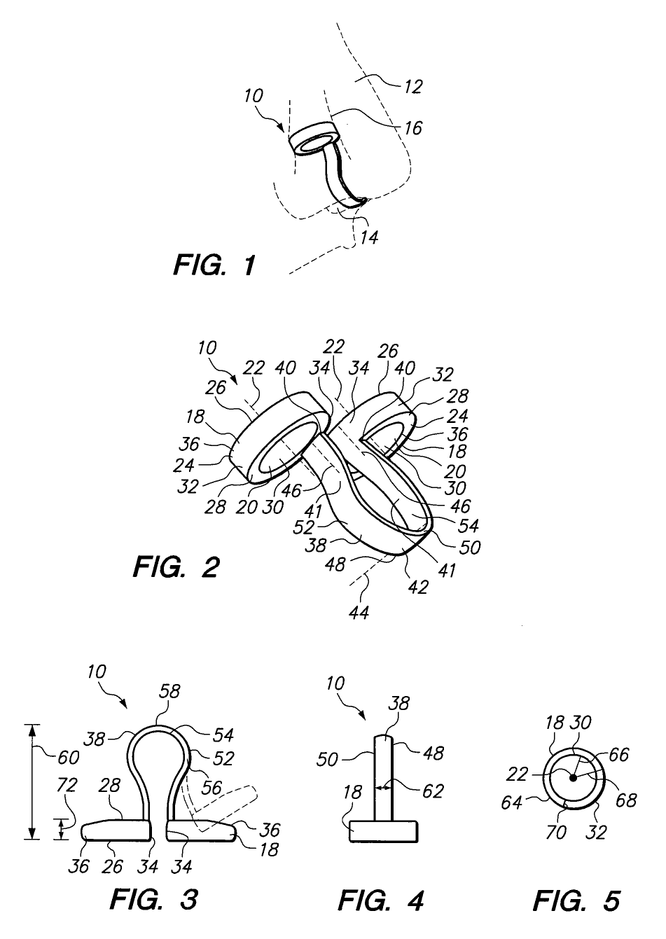

Referring initially to FIG. 1, a nasal dilator for facilitating nasal breathing and eliminating snoring is shown and generally designated 10. As shown in FIG. 1, during use of the nasal dilator 10 by a user 12, the dilator 10 is inserted into the user's nostrils 14. Further, the nasal dilator 10 engages the nasal wall 16 in order to facilitate breathing by user 12.

Referring now to FIG. 2, the structure of the nasal dilator 10 is more clearly seen. As shown, the nasal dilator 10 includes a pair of tubular dilation members 18. Further, each dilation member 18 forms an aperture 20 that defines an aperture axis 22. Also, each dilation member 18 includes a wall 24 that extends in the direction of the axis 22 from a substantially planar distal edge 26 to a proximal edge 28. As shown, the wall 24 of each dilation member 18 has an inner surface 30 and an outer surface 32. It can be seen that each dilation member 18 includes a peri-septum side 34 and an apo-septum side 36.

As further shown in FIG. 2, the nasal dilator 10 includes a substantially U-shaped bridge 38 that is connected to the dilation members 18. Specifically, the bridge 38 extends to two ends 40 that are attached to the peri-septum sides 34 of the dilation members 18. In FIG. 2, it can be seen that the bridge 38 includes two legs 41 that are interconnected by a handle portion 42. In accordance with the present invention, the handle portion 42 defines a flex axis 44 about which the handle portion 42 flexes. Further, each leg 41 defines a leg axis 46 that is substantially parallel to the aperture axis 22 of the connected dilation member 18. As shown, the leg axes 46 form a plane that is substantially perpendicular to the flex axis 44 of the handle portion 42.

Still referring to FIG. 2, it can be seen that the bridge 38 extends in the direction of flex axis 44 from an edge 48 to an edge 50. Further, the bridge 38 includes a surface 52 and a surface 54. Cross-referencing FIG. 2 with FIG. 31 the bridge 38 is shown to have a substantially constant thickness 56 between the surfaces 52 and 54. Preferably, the thickness 56 of the bridge 38 is approximately 1/16 of an inch. Also, the bridge 38 is shown to include an apex 58 in the handle portion 42 at the greatest distance 60 between the surface 52 of the bridge 38 and the distal edges 26 of the dilation members 18. Preferably, the distance 60 is equal to approximately 13/16 of an inch. Now cross-referencing FIG. 2 with FIG. 4, the bridge 38 is shown to have a substantially constant width 62 extending between the edges 48 and 50. In preferred embodiments, the width 62 of the bridge 38 is approximately ⅛ of an inch.

Referring now to FIG. 5, an exemplary dilation member 18 is illustrated. As shown in its substantially cylindrical configuration 64, the dilation member 18 is centered about the aperture axis 22. More specifically, the inner surface 30 of the dilation member 18 is shown to be distanced from the axis 22 by a radius 66 of approximately 5/32 of an inch. Further, the outer surface 32 of the dilation member 18 is distanced from the axis 22 by a radius 68 of approximately 7/32 of an inch. With this structure, the thickness 70 of the dilation member 18 is approximately ⅛ of an inch.

Referring back to FIG. 3, further structural detail of the dilation members 18 are illustrated. For instance, the dilation members 18 include a length 72 in the direction of the axis 22 (shown in FIGS. 2 and 5) between the distal edge 26 and the proximal edge 28. Further, the length 72 is shown to decrease from the peri-septum side 34 to the apo-septum side 36. Specifically, the length 72 varies between approximately 5/32 of an inch and approximately 3/32 of an inch.

In operation, a user 12 holds the handle portion 42 and inserts the dilation members 18 of nasal dilator 10 into the nostrils 14. During insertion, the outer surface 32 of each dilation member 18 engages the wall 16 of each nostril 14. Further, due to the fact that each dilation member 18 is biased toward its cylindrical configuration 64, each dilation member 18 urges each wall 16 radially away from the respective aperture axis 22. Importantly, if the user's nostrils 14 do not correspond to the base shape of the U-shaped bridge 38, the legs 41 pivot about the flex axis 44 (as shown in phantom in FIG. 3). It is noted that the bridge 38 is not strongly biased toward its U-shape so that pressure is not extended against the walls 16 about the flex axis 44. In this manner, the nasal dilator 10 can be used with nostrils 14 having a wide variety of dimensions and features.

After insertion of the dilation members 18 into engagement with the walls 16, the position of the nasal dilator 10 may be further adjusted for comfort. Specifically, the user 12 can grip the handle portion 42 and manipulate the position of the dilation members 18 within the nostrils 14. After a comfortable position is attained, movement of the dilation members 18 about the apertures' axes 22 is prevented through their engagement with the walls 16. Also, non-axial movement of the dilation members 18, i.e., twisting or turning, is prevented by the bridge 38, which allows movement of the dilation members 18 only about the flex axis 44.

While the particular Device for Prevention of Snoring as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A nasal dilator for insertion into a user's nostrils to facilitate breathing therethrough, the nasal dilator comprising:
a substantially U-shaped bridge having a first leg and a second leg interconnected by a handle member, with the first leg terminating at a first end and defining a first leg axis, and with the second leg terminating at a second end and defining a second leg axis, wherein the handle member flexes along a handle axis perpendicular to a plane defined by the leg axes to facilitate insertion of the dilator into the user's nostrils;
a first dilation member attached to the first end of the bridge, with the first dilation member being tubular and forming a first aperture that defines a first aperture axis substantially parallel to the first leg axis, wherein the first dilation member is flexibly biased toward a substantially cylindrical configuration, and wherein, upon insertion into a first nostril, the first dilation member engages a first nasal wall to urge the first nasal wall radially away from the first aperture axis to facilitate breathing through the first nostril; and
a second dilation member attached to the second end of the bridge, with the second dilation member being tubular and forming a second aperture that defines a second aperture axis substantially parallel to the second leg axis, wherein the second dilation member is flexibly biased toward a substantially cylindrical configuration, and wherein, upon insertion into a second nostril, the second dilation member engages a second nasal wall to urge the second nasal wall radially away from the second aperture axis to facilitate breathing through the second nostril.

2. A nasal dilator as recited in claim 1 wherein each dilation member has a peri-septum side and an apo-septum side, and wherein each dilation member is attached to the respective leg at the respective peri-septum side.

3. A nasal dilator as recited in claim 2 wherein each dilation member has a length extending in a direction parallel to the respective aperture axis from a proximal edge to a distal edge, and wherein, for each dilation member, the length decreases from the peri-septum side to the apo-septum side.

4. A nasal dilator as recited in claim 3 wherein each proximal edge is substantially planar.

5. A nasal dilator as recited in claim 3 wherein each dilation member has a thickness extending radially from an inner surface to an outer surface, and wherein the thickness is substantially constant for each dilation member.

6. A nasal dilator as recited in claim 5 wherein the bridge has a width extending in a direction parallel to the handle axis from a first edge to a second edge, and wherein the width is substantially constant through the first leg, handle member and second leg.

7. A nasal dilator as recited in claim 6 wherein the bridge has a thickness extending in the plane defined by the leg axes from a first surface to a second surface, and wherein the thickness is substantially constant through the first leg, handle member and second leg.

8. A nasal dilator as recited in claim 7 wherein the thickness of each dilation member is substantially equal to the width of the bridge.

9. A nasal dilator as recited in claim 8, wherein the bridge has an apex at a greatest distance from the lower surfaces of the dilation members to the first surface of the bridge.

10. A nasal dilator as recited in claim 9 wherein the greatest distance is equal to approximately 13/16 of an inch, the width of the bridge and the thickness of each dilation member is approximately ⅛ of an inch, the thickness of the bridge is approximately 1/16 of an inch, and the length of each dilation member varies between approximately 5/32 and 3/32 of an inch.

11. A nasal dilator for insertion into a user's nostrils to facilitate breathing therethrough, the nasal dilator consisting of:
a first dilation member forming a first aperture defining a first aperture axis, wherein the first dilation member is flexibly biased toward a substantially cylindrical configuration, and wherein, upon insertion into a first nostril, the first dilation member engages a first nasal wall to urge the first nasal wall radially away from the first aperture axis to facilitate breathing through the first nostril;
a second dilation member forming a second aperture defining a second aperture axis, wherein the second dilation member is flexibly biased toward a substantially cylindrical configuration, and wherein, upon insertion into a second nostril, the second dilation member engages a second nasal wall to urge the second nasal wall radially away from the second aperture axis to facilitate breathing through the second nostril; and
a means for connecting the first dilation member and the second dilation member and for preventing non-axial movement of the dilation members after insertion into the respective nostrils.

12. A nasal dilator as recited in claim 11 wherein each dilation member has a peri-septum side and an apo-septum side, and the dilation members are connected via the respective peri-septum sides.

13. A nasal dilator as recited in claim 12 wherein each dilation member has a length extending in a direction parallel to the respective aperture axis from a proximal edge to a distal edge, and wherein, for each dilation member, the length decreases from the peri-septum side to the apo-septum side.

14. A nasal dilator as recited in claim 13 wherein each proximal edge is substantially planar.

15. A nasal dilator as recited in claim 13 wherein each dilation member has a thickness extending radially from an inner surface to an outer surface, and wherein the thickness is substantially constant for each dilation member.

16. A nasal dilator as recited in claim 15 wherein the thickness of each dilation member is approximately 1/16 of an inch, and the length of each dilation member varies between approximately 5/32 and 3/32 of an inch.

17. A nasal dilator as recited in claim 11 wherein said connecting and preventing means consists of a substantially U-shaped bridge having a first leg and a second leg interconnected by a handle member, with the first leg terminating at a first end and defining a first leg axis, and with the second leg terminating at a second end and defining a second leg axis, wherein the first end connects to the first dilation member and the second end connects to the second dilation member.

18. A nasal dilator as recited in claim 17 wherein the handle member flexes along a handle axis perpendicular to a plane defined by the leg axes to facilitate insertion of the dilator into the user's nostrils.

19. A method for facilitating breathing through a user's nostril comprising the steps of:
providing a nasal dilator including a substantially U-shaped bridge having a first leg and a second leg interconnected by a handle member, with the first leg terminating at a first end and defining a first leg axis, and with the second leg terminating at a second end and defining a second leg axis, wherein the handle member flexes along a handle axis perpendicular to a plane defined by the leg axes; with the nasal dilator further including a first dilation member attached to the first end of the bridge, with the first dilation member being tubular and forming a first aperture that defines a first aperture axis substantially parallel to the first leg axis, wherein the first dilation member is flexibly biased toward a substantially cylindrical configuration; and with the nasal dilator further including a second dilation member attached to the second end of the bridge, with the second dilation member being tubular and forming a second aperture that defines a second aperture axis substantially parallel to the second leg axis, wherein the second dilation member is flexibly biased toward a substantially cylindrical configuration;
gripping the handle and inserting the first dilation member into a first nostril and the second dilation member into a second nostril, wherein, upon insertion into the respective nostril, each dilation member engages a respective nasal wall to urge the respective nasal wall radially away from the respective aperture axis to facilitate breathing; and
adjusting the position of each dilation member for the user's comfort by manipulating the handle, with the bridge preventing non-axial movement of each dilation member after the adjusting step.

20. A method as recited in claim 19 wherein each dilation member has a peri-septum side and an apo-septum side, and wherein each dilation member is attached to the respective leg at the respective peri-septum side.
